(19) United States
(12) Patent Application Publication
(10) Pub. No.: US 2002/0035125 A1
(43) Pub. Date: Mar. 21, 2002

(54) Therapeutic combination

(76) Inventor: Charles L. Shear, Gales Ferry, CT (US)

Correspondence Address: Gregg C. Benson, Pfizer Inc., Patent Department, MS 4159, Eastern Point Road, Groton, CT, 06340, US

(21) Appl. No.: 09/929,862

(22) Filed: Aug. 14, 2001

Related U.S. Application Data
(60) Provisional application No. 60/225,238, filed on Aug. 15, 2000.

(51) Int. Cl. A61K 31/47; A61K 31/4015

(52) U.S. Cl. 514/313; 514/422; 514/423

(57) ABSTRACT

Pharmaceutical combinations of a CETP inhibitor and atorvastatin or hydroxy metabolites thereof or a pharmaceutically acceptable salt thereof, methods of using such combinations and kits containing such combinations for the treatment of atherosclerosis, angina, elevated cholesterol and low HDL levels and for the management of cardiac risk.

This application claims priority from provisional application U.S. Ser No. 60/225,238 filed Aug. 15, 2000, the benefit of which is hereby claimed under 37 C.F.R.§1.78(a)(3).

This invention relates to pharmaceutical combinations of cholesterol ester transfer protein (CETP) inhibitors in particular, [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, and atorvastatin and metabolites thereof and pharmaceutically acceptable salts thereof.

### BACKGROUND OF THE INVENTION

[2R,4S]4-[(3,5-Bis-trifluoromethyl-benzyl)-methoxycarbonyl-amino]-2-ethyl-6-trifluoromethyl-3,4-dihydro-2H-quinoline-1-carboxylic acid ethyl ester is disclosed in PCT/IB99/01532 application published as WO 00/17164 on Mar. 30, 2000 as a CETP inhibitor for the elevation of certain plasma lipid levels and to lower certain other plasma lipid levels and accordingly to prevent the occurrence of and treat diseases such as lipid abnormalities, atherosclerosis and cardiovascular diseases. That published application also discloses the combination of a genus of 4-carboxyamino-2-substituted-1,2,3,4-tetrahydroquinolines with a preferred group of 3-hydroxy-3-methylglutaryl-coenzyme A (HMG-CoA) reductase inhibitors being lovastatin, simvastatin, pravastatin, fluvastatin, atorvastatin or rivastatin.

Commonly assigned U.S. provisional application Ser. No. 60/168,051 filed Nov. 30, 1999 discloses crystalline forms of [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, specifically anhydrous and monoethanolate crystalline forms.

Commonly assigned U.S. provisional application Ser. No. 60/167,967 filed Nov. 30, 1999 discloses methods for making [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester.

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are lipid lowering agents.

Atorvastatin calcium, disclosed in U.S. Pat. No. 5,273,995, which is incorporated herein by reference, is currently sold as Lipitor® and has the formula

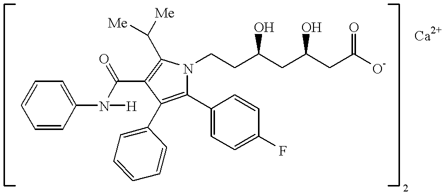

Atorvastatin calcium is a selective, competitive inhibitor of HMG-CoA. As such, atorvastatin calcium is a potent lipid lowering compound. The free carboxylic acid form of atorvastatin exists predominantly as the lactone of the formula

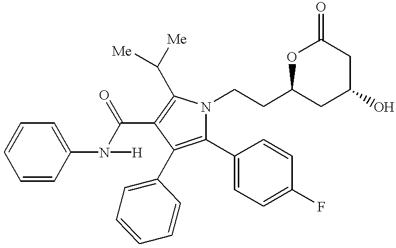

and is disclosed in U.S. Pat. No. 4,681,893, which is incorporated herein by reference.

Hydroxylated derivatives of atorvastatin (hydroxy metabolites) having the formula below wherein R¹ is hydroxy are disclosed in U.S. Pat. No. 5,385,929, the disclosure of which is hereby incorporated by reference.

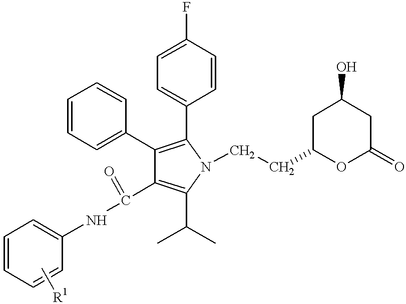

One derivative disclosed in U.S. Pat. No. 5,385,929 is (2R-trans)-5-(4-fluorophenyl)-2-(1-methylethyl)-N-(2-hydroxyphenyl)-4-phenyl-1-[2-(tetrahydro-4-hydroxy-6-oxo-2H-pyran-2-yl)ethyl]-1H-pyrrole-3-carboxamide.

Another derivative disclosed in U.S. Pat. No. 5,385,929 (Example 2) is (2R-trans)-5-(4-fluorophenyl)-2-(1-methylethyl)-N-(3-hydroxyphenyl)-4-phenyl-1-[2-(tetrahydro-4-hydroxy-6-oxo-2H-pyran-2-yl)ethyl]-1H-pyrrole-3-carboxamide.

Yet another derivative disclosed in U.S. Pat. No. 5,385,929 (Example 1) is (2R-trans)-5-(4-fluorophenyl)-2-(1-methylethyl)-N-(4-hydroxyphenyl)-4-phenyl-1-[2-(tetrahydro-4-hydroxy-6-oxo-2H-pyran-2-yl)ethyl]-1H-pyrrole-3-carboxamide.

Atherosclerosis is a condition characterized by irregularly distributed lipid deposits in the intima of arteries, including coronary, carotid and peripheral arteries. Atherosclerotic coronary heart disease (hereinafter termed “CHD”) accounts for 53% of all deaths attributable to a cardiovascular event. CHD accounts for nearly one-half (about $50-60 billion) of the total U.S. cardiovascular healthcare expenditures and about 6% of the overall national medical bill each year. Despite attempts to modify secondary risk factors such as, inter alia, smoking, obesity and lack of exercise, and treatment of dyslipidemia with dietary modification and drug therapy, CHD remains the most common cause of death in the United States.

Risk for development of this condition has been shown to be strongly correlated with certain plasma lipid levels. While elevated LDL-C may be the most recognized form of dyslipidemia, it is by no means the only significant lipid associated contributor to CHD. Low HDL-C is also a known risk factor for CHD (Gordon, D. J., et al.,: “High-density Lipoprotein Cholesterol and Cardiovascular Disease”, Circulation, (1989), 79: 8-15).

High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

Among the many factors controlling plasma levels of these disease dependent principles, cholesteryl ester transfer protein (CETP) activity affects all three. The role of this 70,000 dalton plasma glycoprotein found in a number of animal species, including humans, is to transfer cholesteryl ester and triglyceride between lipoprotein particles, including high density lipoproteins (HDL), low density lipoproteins (LDL), very low density lipoproteins (VLDL), and chylomicrons. The net result of CETP activity is a lowering of HDL cholesterol and an increase in LDL cholesterol. This effect on lipoprotein profile is believed to be pro-atherogenic, especially in subjects whose lipid profile constitutes an increased risk for CHD.

No wholly satisfactory HDL-elevating therapies exist. Niacin can significantly increase HDL, but has serious toleration issues which reduce compliance. Fibrates and the HMG CoA reductase inhibitors raise HDL-C only modestly (˜10-12%). As a result, there is a significant unmet medical need for a well-tolerated agent which can significantly elevate plasma HDL levels, thereby reversing or slowing the progression of atherosclerosis.

High levels of blood cholesterol and blood lipids are conditions involved in the onset of atherosclerosis. It is well known that inhibitors of 3-hydroxy-3-methylglutaryl-coenzyme A reductase (HMG-CoA reductase) are effective in lowering the level of blood plasma cholesterol, especially low density lipoprotein cholesterol (LDL-C), in man (Brown and Goldstein, New England Journal of Medicine, 1981, 305, No. 9, 515-517). It has now been established that lowering LDL-C levels affords protection from coronary heart disease (see, e.g., The Scandinavian Simvastatin Survival Study Group: Randomised trial of cholesterol lowering in 4444 patients with coronary heart disease: the Scandinavian Simvastatin Survival Study (4S), Lancet, 1994, 344, 1383-89; and Shepherd, J. et al., Prevention of coronary heart disease with pravastatin in men with hypercholesterolemia, New England Journal of Medicine, 1995, 333, 1301-07).

Angina pectoris is a severe constricting pain in the chest, often radiating from the precordium to the left shoulder and down the left arm. Often angina pectoris is due to ischemia of the heart and is usually caused by coronary disease.

Currently the treatment of symptomatic angina pectoris varies significantly from country to country. In the U.S., patients who present with symptomatic, stable angina pectoris are frequently treated with surgical procedures or PTCA. Patients who undergo PTCA or other surgical procedures designed to treat angina pectoris frequently experience complications such as restenosis. This restenosis may be manifested either as a short term proliferative response to angioplasty-induced trauma or as long term progression of the atherosclerotic process in both graft vessels and angioplastied segments.

The symptomatic management of angina pectoris involves the use of a number of drugs, frequently as a combination of two or more of the following classes: beta blockers, nitrates and calcium channel blockers. Most, if not all, of these patients require therapy with a lipid lowering agent as well. The National Cholesterol Education Program (NCEP) recognizes patients with existing coronary artery disease as a special class requiring aggressive management of raised LDL-C.

### SUMMARY OF THE INVENTION

This invention is directed to a pharmaceutical composition comprising a therapeutically effective amount of a composition comprising:

a. [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester;

b. atorvastatin or the corresponding cyclized lactone form of atorvastatin, a 2-hydroxy, 3-hydroxy or 4-hydroxy derivative of atorvastatin or the cyclized lactone form of atorvastatin, or a pharmaceutically acceptable salt thereof; and

c. a pharmaceutically acceptable carrier, vehicle or diluent.

As used herein the derivatives (hydroxy metabolites) of the cyclized lactone form of atorvastatin or atorvastatin (the open chain form) described as 2-hydroxy, 3-hydroxy or 4-hydroxy have the Formula I and IA below, respectively,

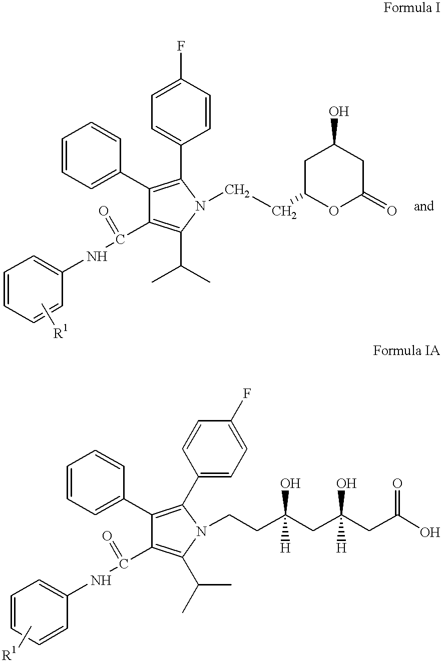

wherein R¹ is hydroxy.

Preferably the composition comprises atorvastatin and it is especially preferred that the composition comprises the hemicalcium salt of atorvastatin.

Preferably R¹ is 2-hydroxy.

This invention is also directed to a method for treating a mammal (e.g., a human either male or female) in need of therapeutic treatment comprising administering to said mammal a therapeutically effective amount of:

(a) a first compound, said first compound being [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester; and

(b) a second compound, said second compound being atorvastatin or the cyclized lactone form of atorvastatin, a 2-hydroxy, 3-hydroxy or 4-hydroxy derivative of said compounds or a pharmaceutically acceptable salt thereof;

wherein said first compound and said second compound are each optionally and independently administered together with a pharmaceutically acceptable carrier, vehicle or diluent.

Preferably the composition comprises atorvastatin and it is especially preferred that the composition comprises the hemicalcium salt of atorvastatin.

Preferably R¹ is 2-hydroxy.

Preferably the first compound and the second compound are administered simultaneously.

Preferably the first compound and the second compound are administered sequentially in either order.

Preferably the therapeutic treatment comprises antiatherosclerotic treatment.

Preferably the therapeutic treatment comprises slowing and/or arresting the progression of atherosclerotic plaques.

Preferably the progression of atherosclerotic plaques is slowed in coronary arteries.

Preferably the progression of atherosclerotic plaques is slowed in carotid arteries.

Preferably the progression of atherosclerotic plaques is slowed in the peripheral arterial system.

Preferably the treatment of atherosclerosis causes the regression of atherosclerotic plaques.

Preferably the regression of atherosclerotic plaques occurs in coronary arteries.

Preferably the regression of atherosclerotic plaques occurs in carotid arteries.

Preferably the regression of atherosclerotic plaques occurs in the peripheral arterial system.

Preferably the therapeutic treatment comprises HDL elevation treatment and antihyperlipidemic treatment (including LDL lowering).

Preferalby the therapeutic treatment comprises antianginal treatment.

Preferably the therapeutic treatment comprises cardiac risk management.

This invention is also directed to a kit for achieving a therapeutic effect in a mammal comprising a therapeutically effective amount of a composition comprising:

a. [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and a pharmaceutically acceptable carrier, vehicle or diluent in a first unit dosage form;

b. atorvastatin or the cyclized lactone form of atorvastatin, a 2-hydroxy, 3-hydroxy or 4-hydroxy derivative of said compounds or a pharmaceutically acceptable salt thereof and a pharmaceutically acceptable carrier, vehicle or diluent in a second unit dosage form; and

c. means for containing said first and second dosage forms.

Preferably the composition comprises atorvastatin and it is especially preferred that the composition comprises the hemicalcium salt of atorvastatin.

Preferably R¹ is 2-hydroxy.

This invention is also particularly directed to a kit wherein the therapeutic effect is the prevention and/or treatment of atherosclerosis.

This invention is still more particularly directed to a kit wherein the treatment of atherosclerosis slows the progression of atherosclerotic plaques.

This invention is further directed to a kit wherein the progression of atherosclerotic plaques is slowed in coronary arteries.

This invention is still further directed to a kit wherein the progression of atherosclerotic plaques is slowed in carotid arteries.

This invention is still further directed to a kit wherein the progression of atherosclerotic plaques is slowed in the peripheral arterial system.

This invention is still further directed to a kit wherein the treatment of atherosclerosis causes the regression of atherosclerotic plaques.

This invention is still further directed to a kit wherein the regression of atherosclerotic plaques occurs in coronary arteries.

This invention is still further directed to a kit wherein the regression of atherosclerotic plaques occurs in carotid arteries.

This invention is still further directed to a kit wherein the regression of atherosclerotic plaques occurs in the peripheral arterial system.

This invention is still more particularly directed to a kit wherein the therapeutic effect is treatment of low HDL levels and hyperlipidemia.

This invention is still more particularly directed to a kit wherein the therapeutic effect is the prevention and/or treatment of angina pectoris.

This invention is also particularly directed to a kit wherein the therapeutic effect is the management of cardiac risk.

This invention is also directed to a first pharmaceutical composition for use with a second pharmaceutical composition for achieving a therapeutic effect in a mammal, which effect is greater than the individual therapeutic effects achieved by administering said first or second pharmaceutical compositions separately and which second pharmaceutical composition comprises an amount of atorvastatin or the cyclized lactone form of atorvastatin, a 2-hydroxy, 3-hydroxy or 4-hydroxy derivative of said compounds or a pharmaceutically acceptable salt thereof and a pharmaceutically acceptable carrier, vehicle or diluent, said first pharmaceutical composition comprising of [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and a pharmaceutically acceptable carrier, vehicle or diluent.

This invention is also directed to a first pharmaceutical composition for use with a second pharmaceutical composition for achieving a therapeutic effect in a mammal, which effect is greater than the individual therapeutic effects achieved by administering said first or second pharmaceutical compositions separately and which second pharmaceutical composition comprises an amount of [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and a pharmaceutically acceptable carrier, vehicle or diluent, said first pharmaceutical composition comprising an amount of atorvastatin or the cyclized lactone form of atorvastatin, a 2-hydroxy, 3 hydroxy or 4-hydroxy derivative of said compounds or a pharmaceutically acceptable salt thereof and a pharmaceutically acceptable carrier, vehicle or diluent.

In the above two pharmaceutical compositions the following are preferred embodiments.

Preferably the therapeutic effect is the prevention and/or treatment of atherosclerosis.

Preferably the therapeutic effect is a LDL-C lowering effect and a HDL-C raising effect in a mammal suffering from hyperlipidemia and low HDL levels.

Preferably the therapeutic effect is the prevention of the occurrence of angina in a mammal at high risk thereof.

Preferably the therapeutic effect is the management of cardiac risk in a mammal at risk of suffering an adverse cardiac event.

Preferably the composition comprises atorvastatin and it is especially preferred that the composition comprises the hemicalcium salt of atorvastatin.

Preferably R¹ is 2-hydroxy.

Preferably the antiatherosclerotic effect is manifested by a slowing of the progression of atherosclerotic plaques.

Preferably the progression of atherosclerotic plaques is slowed in coronary arteries.

Preferably the progression of atherosclerotic plaques is slowed in carotid arteries.

Preferably the progression of atherosclerotic plaques is slowed in the peripheral arterial system.

Preferably the antiatherosclerotic effect is manifested by a regression of atherosclerotic plaques.

Preferably the regression of atherosclerotic plaques occurs in coronary arteries.

Preferably the regression of atherosclerotic plaques occurs in carotid arteries.

Preferably the regression of atherosclerotic plaques occurs in the peripheral arterial system.

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

Where used herein, the term “cardiac risk” means the likelihood that a subject will suffer a future adverse cardiac event such as, e.g., myocardial infarction, cardiac arrest, cardiac failure or cardiac ischaemia. Cardiac risk is calculated using the Framingham Risk Equation. The term “cardiac risk management” means that the risk of future adverse cardiac events is substantially reduced.

As used herein, the expressions “reaction-inert solvent” and “inert solvent” refers to a solvent or a mixture thereof which does not interact with starting materials, reagents, intermediates or products in a manner which adversely affects the yield of the desired product.

A chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

As used herein the term mammals is meant to refer to all mammals which contain CETP in their plasma, for example, rabbits and primates such as monkeys and humans (e.g., male or female). Certain other mammals e.g., dogs, cats, cattle, goats, sheep and horses do not contain CETP in their plasma and so are not included herein.

The term “treating”, “treat” or “treatment” as used herein includes preventative (e.g., prophylactic) and palliative treatment.

By “pharmaceutically acceptable” is meant the vehicle, carrier, diluent, excipients, and/or salt must be compatible with the other ingredients of the formulation, and not deleterious to the recipient thereof.

### DETAILED DESCRIPTION OF THE INVENTION

[2R,4S]4-[(3,5-Bis-trifluoromethyl-benzyl)-methoxycarbonyl-amino]-2-ethyl-6-trifluoromethyl-3,4-dihydro-2H-quinoline-1-carboxylic acid ethyl ester is disclosed in PCT/IB99/01532 application published as WO 00/17164 on Mar. 30, 2000 and may readily be prepared as described therein (see Examples 7 (racemate) and Example 120). Methods for preparation of this compound (and polymorphs thereof) are also disclosed in commonly assigned U.S. provisional applications Ser. Nos. 60/168,051 and 60/168,051 and hereinafter.

### EXAMPLE 1

cis-4-[(3,5-Bis-trifluoromethyl-benzyl)-methoxycarbonyl-amino]-2-ethyl-6-trifluoromethyl-3,4-dihydro-2H-quinoline-1-carboxylic Acid Ethyl Ester:

A solution of cis-4-(3,5-bis-trifluoromethyl-benzylamino)-2-ethyl-6-trifluoromethyl-3,4-dihydro-2H-quinoline-1-carboxylic acid ethyl ester (2.0 g, 3.7 mmol) and pyridine (0.58 g, 7.4 mmol) in 100 mL of dichloromethane was cooled in an ice/water bath as methyl chloroformate (0.87 g, 9.2 mmol) was added slowly. After stirring overnight at room temperature, the reaction mixture was washed twice with a 2N hydrochloric acid solution, dried over magnesium sulfate, filtered and concentrated in vacuo to afford the crude product, which was purified by silica gel chromatography using 5-10% ethyl acetate/hexanes as eluent to afford 1.8 g of the title product. MS m/z 601 (M⁺+1); ¹H NMR (coalescing mixture of conformers, CDCl₃) δ 0.6-0.8 (bm, 3H), 1.2-1.3 (bm, 3H), 1.3-1.5 (bm, 2H), 1.6-1.75 (bm, 1H), 2.1-2.3 (bm, 1H), 3.7-3.9 (bs, 3H), 4.0-4.4 (bm, 4H), 5.0-5.6 (bm, 2H), 7.1 (s, 1H), 7.4-7.6 (bm, 2H), 7.6-7.8 (bm, 3H).

[2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester was prepared in optically enriched form by resolution of the corresponding racemate, or an intermediate in its synthesis, using standard methods.

### EXAMPLE 2

(1-Benzotriazol-1-yl-propyl)-(4-trifluoromethyl-phenyl)-amine

A two liter, four neck flask under nitrogen atmosphere was charged with benzotriazole (36.96 g, 310 mmol, 1.0 equiv) and dry toluene (400 mL). A room temperature solution of 4-(trifluoromethyl)aniline (39.1 mL, 310 mmol, 1.0 equiv) and 50 mL toluene was added over one minute. A room temperature solution of propionaldehyde (24.6 mL, 341 mmol, 1.1 equiv) and 50 mL toluene was then added over 20 minutes. There was an exotherm from 23° C. to 30° C. during this addition. After stirring 24 h, n-heptane (500 mL) was added, and the slurry stirred an additional 1 h. The suspension was filtered, the solids were washed with n-heptane (1×100 mL, then 1×200 mL, and dried. (1-Benzotriazol-1-yl-propyl)-(4-trifluoromethyl-phenyl)-amine was isolated as shiny white needles (81.3 g, 82%). After 24 h, a second crop was isolated from the filtrate (8.7 g, 9%). mp 130-132° C.; ¹H NMR (DMSO-d6, 400 MHz) δ 0.82 (t, 3H, J=7.5 Hz), 2.25 (m, 2H), 6.49 (m, 1H), 6.80 (d, 2H, J=8.7 Hz), 7.35 (m, 3H), 7.50 (m, 1H), 7.88 (d, 1H, J=8.3 Hz), 7.99 (m, 1H), 8.09 (d, 1H, J=8.5 Hz); ¹³C NMR (DMSO-d6, 100 MHz) δ 149.32, 146.19, 131.46, 127.73, 126.8, 125.33 (q, J=270 Hz), 124.44, 119.88, 118.27 (q, J=31.7 Hz), 112.91, 111.56, 71.03, 28.08, 10.29; DEPT spectrum: quaternary carbons δ 149.32, 146.19, 131.46, 125.33, 118.27; CH carbons δ 127.73, 126.8, 124.44, 119.88, 112.91, 111.56, 71.03; CH₂ carbon δ 28.08; CH₃ carbon δ 10.29; IR (drifts) 3292 (s), 3038 (m), 2975 (m), 1621 (s), 1331 (s), 1320 (s), 1114 (vs); Anal. Calcd for C₁₆H₁₅N₄F₃: C, 59.99; H, 4.72; N, 17.49. Found (first crop): C, 60.16; H, 4.74; N, 17.86. Found (second crop): C, 59.97; H, 4.66; N, 17.63.

### EXAMPLE 3

cis-(2-Ethyl-6-trifluoromethyl-1 2,3,4-tetrahydro-quinolin-4-yl)-carbamic Acid Benzyl Ester

A one liter, four neck flask under nitrogen atmosphere was charged with N-vinyl-carbamic acid benzyl ester (27.66 g, 156 mmol, 1.0 equiv) and dry toluene (500 mL). (1-Benzotriazol-1-yl-propyl)-(4-trifluoromethyl-phenyl)-amine (50.0 g, 156 mmol, 1.0 equiv) and p-toluenesulfonic acid monohydrate (297 mg, 1.56 mmol, 0.01 equiv) were added, and the mixture heated to 70° C. After 2 h, the mixture was cooled to room temperature and transfered to a separatory funnel. Ethyl acetate (500 mL) was added. The mixture was washed 1×200 mL 1N NaOH, 1×200 mL H₂O, 1×200 mL brine, and dried (MgSO₄). The mixture was filtered and the solids washed 1×50 mL ethyl acetate. The filtrate was concentrated to approximately 250 mL. 500 mL toluene were added, and the mixture concentrated to approximately 500 mL. 500 mL n-heptane were added, the slurry was stirred 1 h, filtered through a Buchner funnel, and dried. cis-(2-Ethyl-6-trifluoromethyl-1,2,3,4-tetrahydro-quinolin-4-yl)-carbamic acid benzyl ester was isolated as a white powder (45.04 g, 76%): mp 155-157° C.; ¹H NMR (DMSO-d6, 400 MHz) δ 0.92 (t, 3H, J=7.5 Hz), 1.5 (m, 3H), 2.00 (m, 1H), 3.35 (m, 1H), 4.77 (m, 1H), 5.07 (d, 1H, J=12.5 Hz), 5.15 (d, 1H, J=12.5 Hz), 6.35 (s, 1H), 6.61 (d, 1H, J=8.5 Hz), 7.12 (s, 1H), 7.18 (dd, 1H, J=1.9, 8.5 Hz), 7.4 (m, 5H), 7.70 (d, 1H, J=9.1 Hz); ¹³C NMR (DMSO-d6, 100 MHz) δ 157.03, 149.02, 137.79, 128.82, 128.23, 128.03, 125.9 (q, J=270 Hz), 125.06, 123.50, 121.73, 115.2 (q, J=31.7 Hz), 113.33, 65.85, 52.09, 47.83, 34.02, 28.68, 9.93; DEPT spectrum: quaternary carbons δ 157.03, 149.02, 137.79, 125.9, 121.73, 115.2; CH carbons δ 128.82, 128.23, 128.03, 125.06, 123.50, 113.33, 52.09, 47.83; CH₂ carbons δ 65.85, 34.02, 28.68; CH₃ carbon δ 9.93; IR (drifts) 3430 (m), 3303 (s), 2951 (m), 1686 (vs), 1542 (vs), 1088 (vs); MS (APCI+) m/z (rel. intensity) 379 (M+H⁺, 53), 228 (100); Anal. Calcd for C₂₀H₂₁N₂O₂F₃: C, 63.48; H, 5.59; N, 7.40; Found: C, 63.69; H, 6.06, N, 7.36.

### EXAMPLE 4

cis-4-Benzyloxycarbonylamino-2-ethyl-6-trifluoromethyl-3,4-dihydro-2H-quinoline-1-carboxylic Acid Ethyl Ester

A three liter, four neck flask under nitrogen atmosphere was charged with cis-(2-ethyl-6-trifluoromethyl-1,2,3,4-tetrahydro-quinolin-4-yl)-carbamic acid benzyl ester (96.0 g, 254 mmol, 1.0 equiv), dry dichloromethane (720 mL), and dry pyridine (103 mL, 1.27 mol, 5.0 equiv). A solution of ethyl chloroformate (121 mL, 1.27 mol, 5.0 equiv), in dry dichloromethane (240 mL), was added slowly over 4 h. The addition was exothermic and required a reflux condenser. Once the chloroformate addition was complete, the reaction was cooled in an ice bath and 1350 mL 1N NaOH were added. The mixture was stirred 15 min, then transferred to a separatory funnel. The layers were separated and the aqueous extracted 1×1 L dichloromethane. The combined dichloromethane layers were washed 1×1350 mL 1N HCl, 1×1 L saturated aq. NaHCO₃, 1×1 L brine, and dried (Na₂SO₄). The mixture was filtered, and the filtrate concentrated to an orange oil. 570 mL abs. ethanol were added, and the solution was concentrated. The solids were dissolved in 1370 mL abs. ethanol. 570 mL H₂O were added dropwise over 45 min. The resultant thick slurry was stirred 18 h and filtered. The solids were washed with cold 7:3 abs. ethanol/water (1×250 mL, then 1×100 mL) and dried (vac oven, 45° C.) to give cis-4-benzyloxycarbonylamino-2-ethyl-6-trifluoromethyl-3,4-dihydro-2H-quinoline-1-carboxylic acid ethyl ester as a white, crystalline solid (94.54 g, 83%): mp 92-96° C.; ¹H NMR (CDCl₃, 400 MHz) δ 0.84 (t, 3H, J=7.4 Hz), 1.28 (t, 3H, J=7.0 Hz), 1.4 (m, 2H), 1.62 (m, 1H), 2.53 (m, 1H), 4.23 (m, 2H), 4.47 (m, 1H), 4.79 (m, 1H), 5.01 (d, 1H, J=9.2 Hz), 5.18 (m, 2H), 7.4 (m, 5H), 7.5 (m, 2H), 7.57 (m, 1H); ¹³C NMR (CDCl₃, 100 MHz) δ 155.97, 154.43, 139.44, 136.21, 134.33, 128.61, 128.33, 128.22, 126.32 (q, J=31.7 Hz), 126.18, 124.22, 124.19, 124.12 (q, J=273 Hz), 120.74, 120.70, 67.22, 62.24, 53.47, 46.79, 37.75, 28.25, 14.38, 9.78; DEPT spectrum: quaternary carbons δ 155.97, 154.43, 139.44, 136.21, 134.33, 126.32, 124.12; CH carbons δ 128.61, 128.33, 128.22, 126.18, 124.22, 124.19, 120.74, 120.70, 53.47, 46.79; CH₂ carbons δ 67.22, 62.24, 37.75, 28.25; CH₃ carbons δ 14.38, 9.78; IR (drifts) 3304 (s), 3067 (m), 3033 (m), 2982 (m), 2932 (m), 1723 (s), 1693 (s), 1545 (s); MS (APCI+) m/z (rel. intensity) 451 (M+H⁺, 2), 300 (100); Anal. Calcd for C₂₃H₂₅N₂O₄F₃: C, 61.33; H, 5.60; N, 6.22. Found: C, 61.07; H, 5.69; N, 6.22.

### EXAMPLE 5

cis-4-Amino-2-ethyl-6-trifluoromethyl-3,4-dihydro-2H-quinoline-1-carboxylic Acid Ethyl Ester

A one liter, four neck flask under nitrogen atmosphere was charged with cis-4-benzyloxycarbonylamino-2-ethyl-6-trifluoromethyl-3,4-dihydro-2H-quinoline-1-carboxylic acid ethyl ester (40.1 g, 89 mmol, 1.0 equiv), methanol (400 mL), and ammonium formate (14.0 g, 223 mmol, 2.5 equiv). 10% Pd/C, 50% waterwet (4.0 g) was added, and the slurry heated to 40° C. over 1 h. After 1.5 h, the mixture was cooled to room temperature and filtered through Celite®. The cake was washed 2×100 mL methanol. The filtrate was concentrated to approximately 75 mL, transferred to a separatory funnel, and diluted with 400 mL ethyl acetate. The mixture was washed 1×125 mL saturated aq. NaHCO₃, 1×100 mL brine, and dried (Na₂SO₄). The mixture was filtered and the filtrate concentrated to a clear oil. The oil was crystallized from 100 mL n-heptane to give cis-4-amino-2-ethyl-6-trifluoromethyl-3,4-dihydro-2H-quinoline-1-carboxylic acid ethyl ester as a white crystalline solid (26.05 g, 93%): mp 61.5-63.5° C.; ¹H NMR (CDCl₃, 400 MHz) δ 0.79 (t, 3H, J=7.5 Hz), 1.24 (m, 4H), 1.42 (m, 1H), 1.51 (brs, 2H), 1.62 (m, 1H), 2.46 (m, 1H), 3.73 (m, 1H), 4.17 (m, 2H), 4.36 (m, 1H), 7.44 (m, 2H), 7.66 (m, 1H); ¹³C NMR (CDCl₃, 100 MHz) δ 154.6, 139.3, 138.9, 126.3 (q, J=32 Hz), 125.7, 124.3 (q, J=271 Hz), 123.5, 119.8, 61.96, 54.16, 46.91, 41.50, 28.85, 14.38, 9.60; DEPT spectrum: quaternary carbons δ 154.6, 139.3, 138.9, 126.3, 124.3; CH carbons δ 125.7, 123.5, 119.8, 54.16, 46.91; CH₂ carbons δ 61.96, 41.50, 28.85; CH₃ carbons δ 14.38, 9.60; IR (drifts) 3350 (s), 3293 (m), 2972 (s), 1697 (vs); MS (ES+) m/z (rel. intensity) 358 (M+H+CH₃CN⁺, 55), 317 (M+H⁺, 7), 300 (100); Anal. Calcd for C₁₅H₁₉N₂O₂F₃: C, 56.96; H, 6.06; N, 8.86. Found: C, 56.86; H, 6.28; N, 8.82.

### EXAMPLE 6

(−) (2R,4S)-4-Amino-2-ethyl-6-trifluoromethyl-3,4-dihydro-2H-quinoline-1-carboxylic Acid Ethyl Ester hemi-(−)-dibenzoyl-L-tartrate Salt

A one liter flask under nitrogen atmosphere was charged with cis-4-benzyloxycarbonylamino-2-ethyl-6-trifluoromethyl-3,4-dihydro-2H-quinoline-1-carboxylic acid ethyl ester (24.0 g, 75.9 mmol, 1.0 equiv) and (−) dibenzoyl-L-tartaric acid (anhydrous) (27.19 g, 75.9 mmol, 1.0 equiv). 300 mL of approximately 97% ethanol (prepared by adding 10.5 mL H₂O to 500 mL absolute ethanol, mixing, and measuring out 300 mL) was added. The mixture was stirred at room temperature for 18 h, then filtered. The solids were washed 1×48 mL-approximately 97% ethanol, and dried to give (−) (2R,4S)-4-amino-2-ethyl-6-trifluoromethyl-3,4-dihydro-2H -quinoline-1-carboxylic acid ethyl ester hemi-(−)-dibenzoyl-L-tartrate salt as a white crystalline solid (14.77 g, 39%): mp 189.5-191.5° C. (dec); ¹H NMR (DMSO-d6, 400 MHz) δ 0.62 (t, 3H, J=7.3 Hz), 1.16 (t, 3H, J=7.1 Hz), 1.3 (m, 3H), 2.5 (m, 1H), 4.1 (m, 4H), 5.63 (s, 1H, methine proton in DBTA), 7.47 (m, 2H, DBTA aromatic H's), 7.6 (m, 3H, DBTA aromatic H's), 7.68 (s, 1H), 7.95 (m, 2H), 8.2 (br s, NH₃⁺, did not integrate); ¹³C NMR (DMSO-d6, 100 MHz) δ 169.85, 165.53, 154.10, 140.14, 134.59, 133.51, 130.74, 129.69, 128.98, 126.74, 124.82 (q, J=31.7 Hz), 124.69 (q, J=271 Hz), 124.50, 120.90, 74.49, 62.14, 53.51, 45.94, 38.81, 28.23, 14.63, 9.58; DEPT spectrum: quaternary carbons δ 169.85, 165.53, 154.10, 140.14, 134.59, 130.74, 124.82, 124.69; CH carbons δ 133.51, 129.69, 128.98, 126.74, 124.50, 120.90, 74.49, 53.51, 45.94; CH₂ carbons δ 62.14, 38.81, 28.23; CH₃ carbons δ 14.63, 9.58; IR (drifts) 3278 (m), 2400-3100 (broad), 1703 (vs); MS (ES+) m/z (rel. intensity) 358 (M+H+CH₃CN⁺, 55), 317 (M+H⁺, 7), 300 (100); Anal. Calcd for C₁₅H₁₉N₂O₂F₃C₉H₇O₄: C, 58.18; H, 5.29; N, 5.65. Found: C, 57.99; H, 5.15; N, 5.64; Chiral HPLC: mobile phase 950:50:2 n-hexane:2-propanol:HOAc, flow rate 1.50 mL/min, column temp 40° C., chiralpak™ AD 4.6×250 mm, sample concentration approximately 0.5 mg/mL in approximately 1:1 n-hexane:2-propanol. Authentic racemate shows retention times of 7.5 min and 10.0 min. (−) (2R,4S)-4-Amino-2-ethyl-6-trifluoromethyl-3,4-dihydro-2H-quinoline-1-carboxylic acid ethyl ester hemi-(−)-dibenzoyl-L-tartrate salt: 10.0 min, 88.9%, 7.5 min<<1%, 2.0 min (solvent front) 11.1%; [α]_{D}=−153 (c=1.07, CH₃OH).

### EXAMPLE 7

(−)-(2R,4S)-4-(3,5-Bis-trifluoromethyl-benzylamino)-2-ethyl-6-trifluoromethyl-3,4-dihydro-2H-quinoline-1-carboxylic Acid Ethyl Ester Tosylate Salt

(−) (2R,4S)-4-Amino-2-ethyl-6-trifluoromethyl-3,4-dihydro-2H-quinoline-1-carboxylic acid ethyl ester hemi-(−)-dibenzoyl-L-tartrate salt (13.0 g, 26.2 mmol, 1.0 equiv) was suspended in 1,2-dichloroethane (260 mL) in a 500 mL separatory funnel. The mixture was washed 1×65 mL 1N NaOH, 1×65 mL brine, and dried (MgSO₄). The mixture was filtered, concentrated to approximately approximately 80 mL, and transferred to a 250 mL three neck flask. 3,5-Bis(trifluoromethyl)benzaldehyde (4.53 mL, 27.5 mmol, 1.05 equiv) was added, and the mixture stirred 1 h at room temperature under nitrogen atmosphere. Sodium triacetoxyborohydride (11.1 g, 52.4 mmol, 2.0 equiv) was added in one portion, and the white slurry was stirred 18 h. 50 mL 1,2-dichloroethane and 50 mL 2N NaOH were added, and the aqueous layer extracted 2×50 mL 1,2-dichloroethane. The combined organic extracts were washed 1×31 mL 1N HCl, 1×50 mL saturated aq. NaHCO₃, 1×50 mL brine, and dried (Na₂SO₄). The mixture was filtered and concentrated to a clear oil. The oil was dissolved in methanol (71 mL). p-Toluenesulfonic acid monohydrate (5.23 g, 27.5 mmol, 1.05 equiv) was added. After 5 min, 284 mL isopropyl ether was added. The solution was concentrtated to approximately 35 mL, transferred to a 500 mL three neck flask (mech. stirrer), and diluted with 284 mL isopropyl ether. A thick white slurry formed in 10 minutes. After stirring 3 h, the slurry was filtered and the cake washed 2×70 mL isopropyl ether. After drying, (−)-(2R,4S)-4-(3,5-bis-trifluoromethyl-benzylamino)-2-ethyl-6-trifluoromethyl-3,4-dihydro-2H-quinoline-1-carboxylic acid ethyl ester tosylate salt was isolated as a white powder (16.18 g, 86% overall): mp 191-192° C.; ¹H NMR (DMSO-d6, 400 MHz) δ 0.78 (t, 3H, J=7.5 Hz), 1.21 (t, 3H, J=7.0 Hz), 1.5 (m, 3H), 2.24 (s, 3H), 3.08 (m, 1H), 4.17 (m, 2H), 4.41 (m, 1H), 4.50 (m, 2H), 4.79 (m, 1H), 7.04 (d, 2H, J=7.9 Hz), 7.42 (d, 2H, J=7.9 Hz), 7.7 (m, 2H), 7.81 (s, 1H), 8.21 (s, 1H), 8.35 (s, 2H), 9.58 (brs, 1H), 9.83 (brs, 1H); ¹³C NMR (DMSO-d6, 100 MHz) δ 154.00, 145.46, 140.21, 138.39, 135.33, 132.51, 131.62, 130.79 (q, J=33.2 Hz), 128.49, 127.40, 125.82, 125.36, 124.99 (q, J=31.7 Hz), 124.59 (q, J=271 Hz), 123.69 (q, J=273 Hz), 123.44, 120.33, 62.32, 53.99, 53.79, 47.98, 33.30, 28.61, 21.13, 14.63, 9.58; DEPT spectrum: quaternary carbons δ 154.00, 145.46, 140.21, 138.39, 135.33, 130.79, 124.99, 124.59, 123.69; CH carbons δ 132.51, 131.62, 128.49, 127.40, 125.82, 125.36, 123.44, 120.33, 53.99, 53.79; CH₂ carbons δ 62.32, 47.98, 33.30, 28.61; CH₃ carbons δ 21.13, 14.63, 9.58; IR (drifts) 2300-3100 (broad), 2974 (m), 2731 (m), 2620 (m), 2455 (m), 1714 (s), 1621 (m), 1283 (vs), 1169 (vs), 1126 (vs); MS (ES+) m/z (rel. intensity) 584 (M+H+CH₃CN⁺, 100), 543 (M+H⁺, 80); Anal. Calcd for C₂₄H₂₃N₂O₂F₉.C₇H₈O₃S: C, 52.11; H, 4.37; N, 3.92. Found: C, 52.15; H, 4.22; N, 3.69; [α]_{D}=−77.9 (c=1.05, CH₃OH).

### EXAMPLE 8

(−)-(2R,4S)4-[(3,5-Bis-trifluoromethyl-benzyl)-methoxycarbonyl-amino]-2-ethyl-6-trifluoromethyl-3,4-dihydro-2H-quinoline-1-carboxylic Acid Ethyl Ester Mono Ethanolate

Na₂CO₃ (s) (6.75 g, 63.7 mmol, 3.5 equiv) was added to a room temperature solution of (−)-(2R,4S)-4-(3,5-bis-trifluoromethyl-benzylamino)-2-ethyl-6-trifluoromethyl-3,4-dihydro-2H-quinoline-1-carboxylic acid ethyl ester tosylate salt (13.0 g, 18.2 mmol, 1.0 equiv) in dry THF (130 mL). Methyl chloroformate (3.51 mL, 45.5 mmol, 2.5 equiv) was added neat, dropwise over 2 min. After 24 h, the mixture was concentrated to 65 mL, diluted with 260 mL ethyl acetate, and transferred to a separatory funnel. The mixture was washed 1×90 mL 1N HCl (CO₂ evolution), 1×90 mL saturated aq. NaHCO₃, 1×90 mL brine, and dried (MgSO₄). Filtration and concentration of filtrate afforded a clear oil, which was costripped 3×33 mL 2B ethanol. The oil was dissolved in 33 mL 2B ethanol and seeded with a few milligrams of (−)-(2R,4S)-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester mono ethanolate. After stirring 18 h at room temperature, the slurry was filtered and dried to give (−)-(2R,4S)-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester mono ethanolate as a white crystalline powder (8.66 g, 74%): mp 54-58° C.; ¹H NMR (CDCl₃, 400 MHz, 55° C.) δ 0.73 (t, 3H, J=7.0 Hz), 1.20 (t, EtOH), 1.27 (t, 3H, J=7.1 Hz), 1.42 (m, 2H), 1.66 (m, 1H), 2.25 (br s, 1H), 3.67 (q, EtOH), 3.79 (s, 3H), 4.2 (m, 3H), 4.33 (m, 1H), 5.2 (brs, 2H), 7.12 (s, 1H), 7.49 (d, 1H, J=8.3 Hz), 7.57 (d, 1H, J=8.5 Hz), 7.73 (s, 2H), 7.78 (s, 1H); ¹³C NMR (CDCl₃, 400 MHz) δ 157.74, 154.37, 141.73, 140.05, 133.83, 132.14 (q, J=33 Hz), 126.94, 124.49, 123.96 (q, J=273 Hz), 123.13 (q, J=273 Hz), 121.31, 119.17, 62.29, 58.28, 54.42, 53.71, 53.08, 46.67, 37.01, 29.02, 18.29, 14.32, 9.22, (note: the fourth quartet appears to be buried under the δ 126.94 peak, with J approximately 32 Hz); DEPT spectrum: quaternary carbons δ 157.74, 154.37, 141.73, 140.05, 133.83, 132.14, 123.96, 123.13; CH carbons δ 126.94, 124.49, 121.31, 119.17, 54.42, 53.08; CH₂ carbons δ 62.29, 58.28, 46.67, 37.01, 29.02; CH₃ carbons δ 53.71, 18.29, 14.32, 9.22; IR (drifts) 3489 (s), 2974 (s), 2884 (m), 1701 (vs), 1280 (vs), 1131 (vs); MS (ES+) m/z (rel. intensity) 601 (M+H⁺, 100); Anal. Calcd for C₂₆H₂₅N₂O₄F₉.C₂H₆O: C, 52.01; H, 4.83; N, 4.33. Found: C, 51.84; H, 4.54; N, 4.33; chiral HPLC: mobile phase 950:50:2 n-hexane:2-propanol:HOAc, flow rate 1.0 mL/min, 254 nm, chiralpak AD 4.6×250 mm, column temp 40° C., sample concentration approximately 0.5 mg/mL in 90:10 n-hexane:2-propanol, authentic racemate retention times 3.6 and 4.6 min. (−)-(2R,4S)-4-[(3,5-Bis-trifluoromethyl-benzyl)-methoxycarbonyl-amino]-2-ethyl-6-trifluoromethyl-3,4-dihydro-2H-quinoline-1-carboxylic acid ethyl ester mono ethanolate shows 4.6 min, 99.1% and 3.6 min, not detected; [α]_{D}=−93.3 (c=1.08, CH₃OH).

### EXAMPLE 9

Anhydrous (−)-(2R,4S)-4-[(3,5-Bis-trifluoromethyl-benzyl)-methoxycarbonyl-amino]-2-ethyl-6-trifluoromethyl-3,4-dihydro-2H-quinoline-1-carboxylic Acid Ethyl Ester.

A 2.6 g portion of 4(S)-[(3,5-bis-trifluoromethyl-benzyl)-methoxycarbonyl-amino]-2(R)-ethyl-6-trifluoromethyl-3,4-dihydro-2H-quinoline-1-carboxylic acid ethyl ester (a mixture of predominantly amorphous material with traces of ethanolate crystalline form; the title compound was also prepared in an analogous manner starting from pure amorphous or pure ethanolate material) was charged to 13 milliliters of hexanes and heated to effect a solution at about 60° C. The heat was removed and the reaction was allowed to cool to ambient over a one hour period. The reaction was seeded with anhydrous (−)-(2R,4S)-4-[(3,5-bis-trifluoromethyl-benzyl) -methoxycarbonyl-amino]-2-ethyl-6-trifluoromethyl-3,4-dihydro-2H-quinoline-1-carboxylic acid ethyl ester and granulated for eighteen hours under ambient conditions. Alternately, the anhydrous crystals may be prepared from hexanes without seeding. The product was collected by filtration and air dried. The isolated product X-ray pattern matched the calculated powder pattern.

Density: 1.406

Crystal System: Trigonal

Microscopy: Well formed rods and equant (fractured rods) crystals demonstrating high birefringence when viewed across the C axis. Being in the Trigonal crystal system the crystals do not demonstrate birefringence when viewed down the C axis. The crystals demonstrate a cleavage plane perpendicular to the C axis.

Fusion Microsocopy:

In Type A oil—dissolution at 50° C.

Dry—clear melt at 86° C.

NMR: No trace of ethanolate

Degree of crystallinity: Highly crystalline

Hygroscopicity: Non-hygroscopic at 100% relative humidity over 48 hours.

Appearance: Free flowing white powder.

### EXAMPLE 10

Monoethanolate, (−)-(2R,4S)-4-[(3,5-Bis-trifluoromethyl-benzyl)-methoxycarbonyl-amino]-2-ethyl-6-trifluoromethyl-3,4-dihydro-2H-quinoline-1-carboxylic Acid Ethyl Ester.

4.0 grams of (−)-(2R,4S)-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester were dissolved in 3.5 ml ethanol and sonicated for two minutes to complete dissolution. A white solid formed to which 10 ml ethanol was added and stirred at ambient temperature overnight. A white powder was filtered and collected on 0.22 μm LS filter paper followed by washing with about 15 ml. ethanol. The isolated product X-ray pattern matched the calculated powder pattern.

Density: 1.402

Crystal System: orthorhombic

Microscopy: crystalline needles with moderate birefringence.

Fusion Microsocopy:

In Type A oil—melt and dissolution at 43° C. with loss of water

Dry—clear melt at 43° C.

NMR: shows ethanol of solvation

Degree of crystallinity: highly crystalline

Hygroscopicity: non-hygroscopic

Appearance: free-flowing white power

### EXAMPLE 11

Anhydrous (−)-(2R,4S)-4-[(3,5-bis-trifluromethylbenzyl)-methoxycarbonyl-amino]-2-ethyl-6-trifluoromethyl-3,4-dihydro-2H-quinoline-1-carboxylic Acid Ethyl Ester.

A crude solution of approximately 42 g of (−)-(2R,4S)-4-[(3,5-bis-trifluromethylbenzyl)-methoxycarbonyl-amino]-2-ethyl-6-trifluoromethyl-3,4-dihydro-2H-quinoline-1-carboxylic acid ethyl ester in 500 ml of ethyl acetate (obtained via the process described in Example 8) was concentrated under vacuum to a volume of 100-135 ml. The remaining ethyl acetate was displaced with 3×220 ml 2B EtOH to a final volume of 100-135 ml. This solution was seeded with a crystal of anhydrous (−)-(2R,4S)-4-[(3,5-bis-trifluromethylbenzyl)-methoxycarbonyl-amino]-2-ethyl-6-trifluoromethyl-3,4-dihydro-2H-quinoline-1-carboxylic acid ethyl ester. After stirring 18 hr at room temperature the slurry was filtered and vacuum dried to give 19.81 g of anhydrous (−)-(2R,4S)-4-[(3,5-bis-trifluromethylbenzyl)-methoxycarbonyl-amino]-2-ethyl-6-trifluoromethyl-3,4-dihydro-2H-quinoline-1-carboxylic acid ethyl ester. The melting point behaviour was the same as the material prepared via Example 9 confirming the anhydrous nature of the material.

Atorvastatin or its cyclized lactone form may readily be prepared as described in U.S. Pat. No. 4,681,892, which is incorporated herein by reference. The hemicalcium salt of atorvastatin, which is currently sold as Lipitor®, may readily be prepared as described in U.S. Pat. No. 5,273,995, which is incorporated herein by reference.

The hydroxylated derivatives (metabolites) of atorvastatin (or the cyclized lactone form or pharmaceutically acceptable salts thereof) may be prepared as described in U.S. Pat. No. 5,385,929. The ortho, meta and para hydroxy derivatives are encompassed herein:

(2R-trans)-5-(4-fluorophenyl)-2-(1-methylethyl)-N-(2-hydroxyphenyl)-4-phenyl-1-[2-(tetrahydro-4-hydroxy-6-oxo-2H-pyran-2-yl)ethyl]-1H-pyrrole-3-carboxamide.

(2R-trans)-5-(4-fluorophenyl)-2-(1-methylethyl)-N-(3-hydroxyphenyl)-4-phenyl-1-[2-(tetrahydro-4-hydroxy-6-oxo-2H-pyran-2-yl)ethyl-]1H-pyrrole-3-carboxamide; and

(2R-trans)-5-(4-fluorophenyl)-2-(1-methylethyl)-N-(4-hydroxyphenyl)-4-phenyl-1-[2-(tetrahydro-4-hydroxy-6-oxo-2H-pyran-2-yl)ethyl]-1H-pyrrole-3-carboxamide.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g. sodium and potassium), alkaline earth metal salts (e.g. calcium and magnesium), aluminum salts, ammonium salts, and salts with organic amines such as benzathine (N,N-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

Other pharmaceutically-acceptable cationic salts of atorvastatin may be readily prepared by reacting the free acid form of atorvastatin with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate) and employing a solvent (e.g., ethyl acetate) from which the desired cationic salt precipitates. The salts may also be isolated by concentrating the reaction solution and/or by adding a non-solvent.

The acid addition salts of atorvastatin may be readily prepared by reacting the free base form of atorvastatin with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

In addition, [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester can exist as a monoethanolate and an anhydrous form as described in provisional U.S. application Ser. No. 60/167,967 and such forms are within the scope of the invention.

Atorvastatin, or the cyclized lactone form the ortho, meta and para hydroxy derivatives of said compounds and pharmaceutically acceptable salts thereof may occur as hydrates or solvates. Said hydrates and solvates are also within the scope of the invention.

The pharmaceutical combinations and methods of this invention are all adapted to therapeutic use as agents in the treatment of atherosclerosis, angina pectoris, and a condition characterized by the presence of both low HDL levels and hyperlipidemia in mammals, particularly humans. Further, since these diseases and conditions are closely related to the development of cardiac disease and adverse cardiac conditions, these combinations and methods, by virtue of their action as antiatherosclerotics, antianginals and antihyperlipidemics, are useful in the management of cardiac risk as well as mixed lipid disorders, such as those seen in diabetes and other metabolic syndromes.

The utility of the compounds of the present invention as medical agents in the treatment of atherosclerosis in mammals (e.g. humans) is demonstrated by the activity of the compounds of this invention in conventional assays and the clinical protocol described below:

In the following protocols reference to a CETP inhibitor X is to [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester.

### Atherosclerosis Protocol

This study is a prospective randomized evaluation of the effect of a combination of CETP inhibitor X and atorvastatin (or its metabolities) on the progression/regression of atherosclerotic disease. The study is used to show that a combination of CETP inhibitor X and atorvastatin (or its metabolities) are effective in slowing or arresting the progression or causing regression of existing atherosclerotic disease as evidenced by changes in plaque and/or lumen parameters via various imaging techniques, coronary angiography or carotid ultrasound, in subjects with or without established disease.

This study is an imaging documentation of atherosclerotic disease carried out as a double-blind trial of a minimum of about 500 subjects and preferably of about 780 to about 1200 subjects. It is especially preferred to study about 1200 subjects in this study. Subjects are admitted into the study after satisfying certain entry criteria set forth below.

Entry Criteria:

Subjects accepted for entry into this trial must satisfy certain criteria. Thus the subject must be an adult, either male or female, aged 18-80 years of age in whom cardiovascular imaging is clinically indicated. Subjects will have evidence of atherosclerotic disease that is judged not likely to require intervention over the next 3 years. It is required that the vessels undergoing analysis have not been interfered with. Since percutaneous transluminal cardiac angioplasty (PTCA) interferes with segments by the insertion of a balloon catheter, non-PTCA segments are required for analysis. It is also required that the vessels to be analyzed have not suffered a thrombotic event, such as a myocardial infarct (MI). Thus the requirement for non-MI vessels. Potential areas to be analyzed include: left main, proximal, mid and distal left anterior descending, first and second diagonal branch, proximal and distal left circumflex, first or largest space obtuse marginal, proximal, mid and distal right coronary artery.

Generally, due to the number of patients and the physical limitations of any one facility, the study is carried out at multiple sites. At entry into the study, subjects undergo quantitative coronary as well as carotid artery and/or peripheral vessel imaging at designated testing centers. This establishes baselines for each subject. Once admitted into the test, subjects are randomized to receive either CETP inhibitor X (10-100 mgs) with atorvastatin calcium (10-80 mgs) or its metabolites (0.02 mg/kg-200 mg/kg), each one separately, and/or neither. All doses set forth in this protocol are per day doses. The amount of CETP inhibitor X or atorvastatin (or its metabolites) may be varied as required.

The subjects are monitored for a one to three year period, generally three years being preferred. Imaging assessment of vessels that does not require an invasive procedure are performed at regular intervals throughout the study.

Generally, six month intervals are suitable. Typically this assessment is performed using B-mode ultrasound and/or equivalent equipment. However, a person skilled in the art may use other methods of performing this assessment. Invasive imaging is performed at the conclusion of the one to three year treatment period. The baseline and post-treatment images are evaluated for new lesions or progression of existing atherosclerotic lesions.

The primary objective of this study is to show that the combination of CETP inhibitor X and atorvastatin (or metabolites thereof) or pharmaceutically acceptable salts thereof reduces the progression of atherosclerotic lesions as measured by quantitative coronary angiography (QCA) or CBCT, or IVVS in subjects with clinical coronary artery disease. These techniques measure the amount of atherosclerosis in a vessel.

The primary endpoint of the study is the change in atherosclerotic burden of the affected vessel. Using QCA as an example, the diameter of an arterial segment is measured at various portions along the length of that segment. The average diameter of that segment is then determined. After the average segment diameter of many segments has been determined, the average of all segment averages is determined to arrive at the average mean segment diameter. The mean segment diameter of subjects taking atorvastatin (or its metabolites) or pharmaceutically acceptable salts thereof and the CETP inhibitor X will decline more slowly, will be halted completely, or there will be an increase in the mean segment diameter. These results represent slowed progression of atherosclerosis, halted progression of atherosclerosis and regression of atherosclerosis, respectively.

The secondary objective of this study is to show that the combination of the CETP inhibitor X and atorvastatin (or its metabolites) or a pharmaceutically salt thereof reduces the rate of progression of atherosclerosis in other arteries. For example, using carotid arteries as an example, as measured by the slope of the maximum intimal-medial thickness measurements averaged over 12 separate wall segments (Mean Max) as a function of time, more than does the CETP inhibitor X or atorvastatin (or its metabolites) or pharmaceutically acceptable salt thereof, alone. The intimal-medial thickness of subjects taking atorvastatin (or its metabolites) or pharmaceutically acceptable salt thereof and the CETP inhibitor X will increase more slowly, will cease to increase or will decrease. These results represent slowed progression of atherosclerosis, halted progression of atherosclerosis and regression of atherosclerosis, respectively.

The utility of the compounds of the present invention as medical agents in the treatment of angina pectoris in mammals (e.g., humans) is demonstrated by the activity of the compounds of this invention in conventional assays and the clinical protocol described below:

### Angina Protocol

This study is a double blind, parallel arm, randomized study to show the effectiveness of the CETP inhibitor X and atorvastatin (or its metabolites) or pharmaceutically acceptable salts thereof given in combination in the treatment of symptomatic angina.

Entry Criteria:

Subjects are males or females between 18 and 80 years of age with a history of typical chest pain associated with one of the following objective evidences of cardiac ischemia: (1) stress test segment elevation of about one millimeter or more from the ECG; (2) positive treadmill stress test; (3) new wall motion abnormality on ultrasound; or (4) coronary angiogram with a significant qualifying stenosis. Generally a stenosis of about 30-50% is considered to be significant.

Each subject is evaluated for about ten to thirty-two weeks. At least ten weeks are generally required to complete the study. Sufficient subjects are used in this screen to ensure that about 200 to 800 subjects and preferably about 400 subject are evaluated to complete the study. Subjects are screened for compliance with the entry criteria, set forth below, during a four week run in phase. After the screening criteria are met, subjects are washed out from their current anti-anginal medication and stabilized on a long acting nitrate such as, for example, nitroglycerin, isosorbide-5-mononitrate or isosorbide dinitrate. The term “washed out”, when used in connection with this screen, means the withdrawal of current anti-anginal medication so that substantially all of said medication is eliminated from the body of the subject. A period of eight weeks is preferably allowed for both the wash out period and for the establishment of the subject on stable doses of said nitrate. Subjects having one or two attacks of angina per week while on stable doses of long acting nitrate are generally permitted to skip the wash out phase. After subjects are stabilized on nitrates, the subjects enter the randomization phase provided the subjects continue to have either one or two angina attacks per week. In the randomization phase, the subjects are randomly placed into one of the four arms of the study set forth below. After completing the wash out phase, subjects in compliance with the entry criteria undergo twenty four hour ambulatory electrocardigram (ECG) such as Holter monitoring, exercise stress testing such as a treadmill and evaluation of myocardial perfusion using PET (photon emission tomography) scanning to establish a baseline for each subject. When conducting a stress test, the speed of the treadmill and the gradient of the treadmill can be controlled by a technician. The speed of the treadmill and the angle of the gradient are generally increased during the test. The time intervals between each speed and gradient increase is generally determined using a modified Bruce Protocol.

After the baseline investigations have been completed, subjects are initiated on one of the following four arms of the study: (1) placebo; (2) atorvastatin calcium (about 2.5 mg to about 160 mg) or its metabolites (0.02 mg/kg-200 mg/kg); (3) CETP inhibitor X (about 10 mg to about 120 mg); or (4) a combination of the above doses of CETP inhibitor X and atorvastatin calcium (or its metabolites) together. The subjects are then monitored for two to twenty-four weeks.

After the monitoring period has ended, subjects will undergo the following investigations: (1) twenty four hour ambulatory ECG, such as Holter monitoring; (2) exercise stress testing (e.g. treadmill using said modified Bruce Protocol); and (3) evaluation of myocardial perfusion using PET scanning. Patients keep a diary of painful ischemic events and nitroglycerine consumption. It is generally desirable to have an accurate record of the number of anginal attacks suffered by the patient during the duration of the test. Since a patient generally takes nitroglycerin to ease the pain of an anginal attack, the number of times that the patient administers nitroglycerine provides a reasonably accurate record of the number of anginal attacks.

To demonstrate the effectiveness of the compound combination of this invention, and to determine the dosage amounts of the compound combination of this invention, the person conducting the test will evaluate the subject using the tests described. Successful treatment will yield fewer instances of ischemic events as detected by ECG, will allow the subject to exercise longer or at a higher intensity level on the treadmill, or to exercise without pain on the treadmill, or will yield better perfusion or fewer perfusion defects on photoemission tomography (PET).

The utility of the compounds of the present invention as medical agents in the treatment of lipid abnormalities in mammals (e.g., humans) suffering from a combination of low HDL-C and high LDL-C is demonstrated by the activity of the compounds of this invention in conventional assays and the clinical protocol described below:

### Dyslipidemia Protocol

This study is a double blind, parallel arm, randomized study to show the effectiveness of CETP inhibitor X and atorvastatin calcium (or its metabolites) or pharmaceutically acceptable salts thereof given in combination in controlling both low HDL-C and high LDL-C in subjects who have mild, moderate, or severe levels of these lipid abnormalities.

Each subject is evaluated for 10 to 20 weeks and preferably for 14 weeks. Sufficient subjects are used in this screen to ensure that about 400 to 800 subjects are evaluated to complete the study.

Entry Criteria:

Subjects are male or female adults between 18 and 80 years of age having both low HDL-C and high LDL-C. The presence of these abnormalities is evidenced by evaluation of the low density lipoprotein (LDL) level of the subject relative to certain positive risk factors and evaluation of their HDL-C levels. If the subject has no coronary heart disease (CHD) and has less than two positive risk factors, then the subject is considered to have high LDL if the LDL of the subject is greater than or equal to 190 mg/dl. If the subject has no CHD and has two or more positive risk factors, then the subject is considered to have hyperlipidemia if the LDL of the subject is greater than or equal to 160 mg/dl. If the subject has CHD, then the subject is considered to have hyperlipidemia if the LDL of the subject is greater than or equal to 130.

Positive risk factors include (1) male over 45, (2) female over 55 wherein said female is not undergoing hormone replacement therapy (HRT), (3) family history of premature cardiovascular disease, (4) the subject is a current smoker, (5) the subject has diabetes, (6) an HDL of less than 35, and (7) the subject has hypertension. An HDL of greater than 60 is considered a negative risk factor and will offset one of the above mentioned positive risk factors.

The presence of low HDL is evidenced by a level less than 35 mg/dl.

Subjects are screened for compliance with the entry criteria set forth above. After all screening criteria are met, subjects are washed out from their current lipid lowering medication and are placed on the NCEP ATP II Step 1 diet. The NCEP ATP 11 (adult treatment panel, 2nd revision) Step 1 diet sets forth the amount of saturated and unsaturated fat which can be consumed as a proportion of the total caloric intake. The term “washed out” where used in connection with this screen, means the withdrawal of current lipid lowering medication so that substantially all of said medication is eliminated from the body of the subject. Newly diagnosed subjects generally remain untreated until the test begins. These subjects are also placed on the NCEP Step 1 diet. After the four week wash out and diet stabilization period, subjects undergo the following baseline investigations: (1) medical history and (2) fasting lipid screen. The fasting lipid screen determines baseline lipid levels in the fasting state of a subject. Generally, the subject abstains from food for twelve hours, at which time lipid levels are measured.

After the baseline investigations are performed subjects are started on one of the following: (1) a fixed dose of CETP inhibitor X, generally about 10 to 120 mg; (2) a fixed dose of atorvastatin calcium, generally about 10 to 80 mg or its metabolites (0.02 mg/kg-200 mg/kg); or (3) a combination of the above doses of CETP inhibitor X and atorvastatin calcium (or its metabolites) together. Subjects remain on these doses for a minimum of six weeks, and generally for no more than eight weeks. The subjects return to the testing center at the conclusion of the six to eight weeks so that the baseline evaluations can be repeated. The lipid screen measures the total cholesterol, LDL-cholesterol, HDL-cholesterol, triglycerides, apoB, VLDL (very low density lipoprotein) and other components of the lipid profile of the subject. Improvements in the values obtained after treatment relative to pretreatment values indicate the utility of the compound combination.

The utility of the compounds of the present invention as medical agents in the management of cardiac risk in mammals (e.g., humans) at risk for an adverse cardiac event is demonstrated by the activity of the compounds of this invention in conventional assays and the clinical protocol described below:

### Risk of Future Cardiovascular Events

This study is a double blind, parallel arm, randomized study to show the effectiveness of the CETP inhibitor X and atorvastatin (and its metabolites) or pharmaceutically acceptable salts thereof given in combination in reducing the overall calculated risk of future events in subjects who are at risk for having future cardiovascular events. This risk is calculated by using the Framingham Risk Equation. A subject is considered to be at risk of having a future cardiovascular event if that subject is more than one standard deviation above the mean as calculated by the Framingham Risk Equation. The study is used to evaluate the efficacy of a fixed combination of the CETP inhibitor X and atorvastatin (or its metabolites) in controlling cardiovascular risk by controlling both low HDL and high LDL in patients who have both mild to moderate abnormalities in these lipids.

Each subject is evaluated for 10 to 20 weeks and preferably for 14 weeks. Sufficient subjects are recruited to ensure that about 400 to 800 subjects are evaluated to complete the study.

Entry Criteria:

Subjects included in the study are male or female adult subjects between 18 and 80 years of age with a baseline five year risk which risk is above the median for said subject's age and sex, as defined by the Framingham Heart Study, which is an ongoing prospective study of adult men and women showing that certain risk factors can be used to predict the development of coronary heart disease. The age, sex, systolic and diastolic blood pressure, smoking habit, presence or absence of carbohydrate intolerance, presence or absence of left ventricular hypertrophy, serum cholesterol and high density lipoprotein (HDL) of more than one standard deviation above the norm for the Framingham Population are all evaluated in determining whether a patient is at risk for adverse cardiac event. The values for the risk factors are inserted into the Framingham Risk equation and calculated to determine whether a subject is at risk for a future cardiovascular event.

Subjects are screened for compliance with the entry criteria set forth above. After all screening criteria are met, patients are washed out from their current lipid lowering medication and any other medication which will impact the results of the screen. The patients are then placed on the NCEP ATP II Step 1 diet, as described above. Newly diagnosed subjects generally remain untreated until the test begins. These subjects are also placed on the NCEP ATP II Step 1 diet. After the four week wash out and diet stabilization period, subjects undergo the following baseline investigations: (1) blood pressure; (2) fasting; (3) lipid screen; (4) glucose tolerance test; (5) ECG; and (6) cardiac ultrasound. These tests are carried out using standard procedures well known to persons skilled in the art. The ECG and the cardiac ultrasound are generally used to measure the presence or absence of left ventricular hypertrophy.

After the baseline investigations are performed patients will be started on one of the following: (1) a fixed dose of CETP inhibitor X (about 10-120 mg); (2) a fixed dose of atorvastatin (about 10 to 80 mg) or its metabolites (0.02 mg/kg-200 mg/kg); or (3) the combination of the above doses of CETP inhibitor X and atorvastatin (or its metabolites). Patients are kept on these doses and are asked to return in six to eight weeks so that the baseline evaluations can be repeated. At this time the new values are entered into the Framingham Risk equation to determine whether the subject has a lower, greater or no change in the risk of future cardiovascular event. The above assays demonstrating the effectiveness of [2R,4S]4-[(3,5-bis-trifluoromethyl-benzyl) -methoxycarbonyl-amino]-2-ethyl-6-trifluoromethyl-3,4-dihydro-2H-quinoline-1-carboxylic acid ethyl ester and atorvastatin or hydroxy derivatives thereof or pharmaceutically acceptable salts thereof in the prevention and/or treatment of angina pectoris, atherosclerosis, low HDL and high LDL together, and the management of cardiac risk, also provide a means whereby the activities of the compounds of this invention can be compared between themselves and with the activities of other known compounds. The results of these comparisons are useful for determining dosage levels in mammals, including humans, for the prevention and/or treatment of such diseases.

In general, [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester is administered in a dosage in the range of about 0.1 to about 10 mg/kg/day preferably about 0.5 to about 5 mg/kg/day.

In general atorvastatin or the cyclized lactone form or its pharmaceutically acceptable salts, is administered in a dosage of about 2.5 mg/day to about 160 mg/day. Preferably, atorvastatin calcium is administered in a dosage of about 10 mg/day to about 80/mg day. Typically the hydroxy metabolites of these compounds are administered in a dosage of about 0.02 mg/kg/day-200 mg/kg/day. These dosages being based on an average human subject having a weight of about 65 to about 70 kg.

The compounds of the present invention are generally administered in the form of a pharmaceutical composition comprising at least one of the compounds of this invention together with a pharmaceutically acceptable carrier, vehicle or diluent. Thus, the compounds of this invention can be administered either individually or together in any conventional oral, parenteral or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

The combination of this invention may also be administered in a controlled release formulation such as a slow release or a fast release formulation. Such controlled release dosage formulations of the combination of this invention may be prepared using methods well known to those skilled in the art. The method of preferred administration will be determined by the attendant physician or other person skilled in the art after an evaluation of the subject's condition and requirements. The generally preferred formulation of atorvastatin is Lipitor®. For [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester a generally preferred formulation is a dosage unit form in a capsule, for example a gel capsule, it may contain, in addition to or instead of materials of the above type, a liquid carrier such as a fatty glyceride or mixtures of fatty glycerides, such as olive oil, or Miglyol TM or Capmul TM glycerides. Dosage forms may also include oral suspensions.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the condition or disease of the subject being treated.

Since the present invention relates to the treatment of diseases and conditions with a combination of active ingredients which may be administered separately, the invention also relates to combining separate pharmaceutical compositions in kit form. The kit includes two separate pharmaceutical compositions: [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and atorvastatin (or its metabolites) or a pharmaceutically acceptable salt thereof. The kit includes means for containing the separate compositions such as a divided bottle or a divided foil packet, however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

It should be understood that the invention is not limited to the particular embodiments described herein, but that various changes and modifications may be made without departing from the spirit and scope of this novel concept as defined by the following claims.

1. A pharmaceutical composition comprising a therapeutically effective amount of a composition comprising:
a. [2R,4S]4-[(3, 5-bis-trifluoromethyl-benzyl)-methoxycarbonyl-amino]-2-ethyl-6-trifluoromethyl-3,4-dihydro-2H-quinoline-1-carboxylic acid ethyl ester;
b. a compound of the Formula I 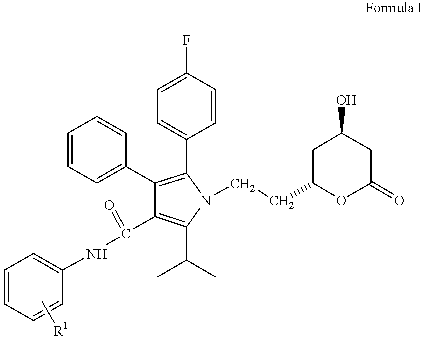
or, the open chain Formula IA 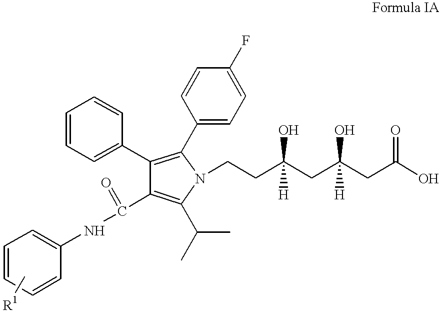
wherein R¹ is hydrogen or hydroxy or the pharmaceutically acceptable salts thereof; and
c. a pharmaceutically acceptable carrier, vehicle or diluent.

2. A pharmaceutical composition as recited in claim 1 wherein R¹ is hydrogen or a pharmaceutically acceptable salt thereof.

3. A pharmaceutical composition as recited in claim 2 comprising the hemicalcium salt of atorvastatin.

4. A pharmaceutical composition as recited in claim 1 wherein R¹ is 2-hydroxy or a pharmaceutically acceptable salt thereof.

5. A method for treating a mammal in need of therapeutic treatment comprising administering to said mammal a therapeutically effective amount of:
a a first compound, said first compound being [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester; and
b. a second compound, said second compound being a compound having the Formula I 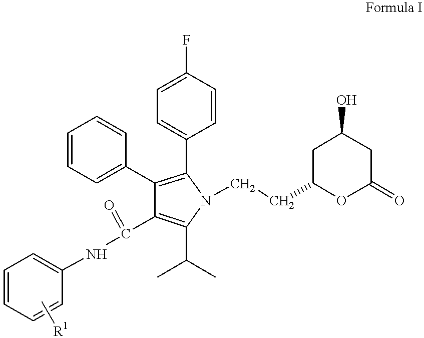
or, the open chain Formula IA 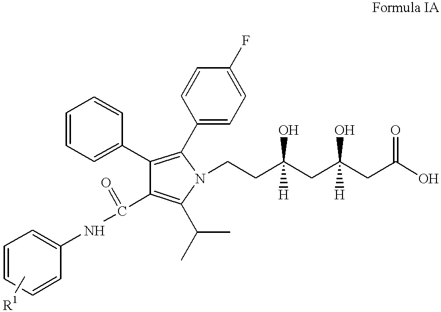
wherein R¹ is hydrogen or hydroxy or the pharmaceutically acceptable salts thereof; and
wherein said first compound and said second compound are each optionally and independently administered together with a pharmaceutically acceptable carrier, vehicle or diluent.

6. A method of treating a mammal as recited in claim 5 wherein R¹ is hydrogen or a pharmaceutically acceptable salt thereof.

7. A method of treating a mammal as recited in claim 6 comprising the hemicalcium salt of atorvastatin.

8. A method of treating a mammal as recited in claim 5 wherein R¹ is 2-hydroxy or a pharmaceutically acceptable salt thereof.

9. A method of treating a mammal as recited in claim 5 wherein atherosclerosis is prevented or treated.

10. A method of treating a mammal as recited in claim 5 wherein the progression of atherosclerotic plaques is slowed.

11. A method of treating a mammal as recited in claim 10 wherein the treatment of atherosclerosis causes the regression of atherosclerotic plaques.

12. A method of treating a mammal as recited in claim 5 wherein the therapeutic treatment comprises HDL elevation treatment and antihyperlipidemic treatment.

13. A method of treating a mammal as recited in claim 5 wherein angina is prevented.

14. A method of treating a mammal as recited in claim 5 wherein the therapeutic treatment comprises cardiac risk management.

15. A kit for achieving a therapeutic effect in a mammal comprising a therapeutically effective amount of a composition comprising:
a. [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and a pharmaceutically acceptable carrier, vehicle or diluent in a first unit dosage form;
b. a compound having the Formula I 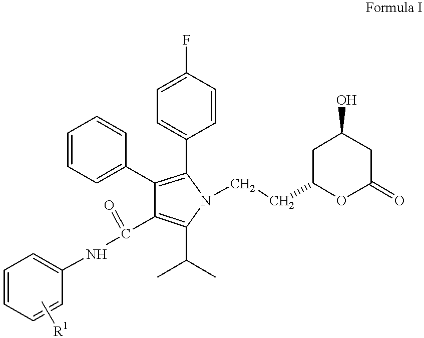
or, the open chain Formula IA 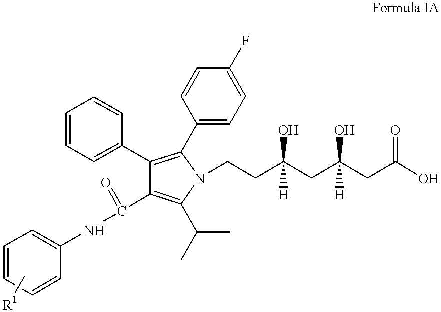
wherein R¹ is hydrogen or hydroxy or the pharmaceutically acceptable salts thereof
and a pharmaceutically acceptable carrier, vehicle or diluent in a second unit dosage form; and
c. means for containing said first and second dosage forms.

16. A kit as recited in claim 15 wherein R¹ is hydrogen or a pharmaceutically acceptable salt thereof.

17. A kit as recited in claim 16 comprising the hemicalcium salt of atorvastatin.

18. A kit as recited in claim 15 wherein R¹ is 2-hydroxy or a pharmaceutically acceptable salt thereof.

19. A first pharmaceutical composition for use with a second pharmaceutical composition for achieving a therapeutic effect in a mammal, which effect is greater than the individual therapeutic effects achieved by administering said first or second pharmaceutical compositions separately and which second pharmaceutical compositions comprises an amount of a Formula I or IA compound or a pharmaceutically acceptable salt thereof having the Formula I 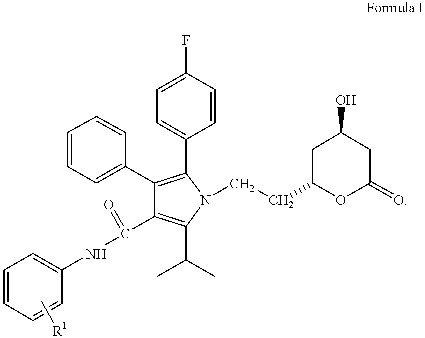
or, the open chain Formula IA 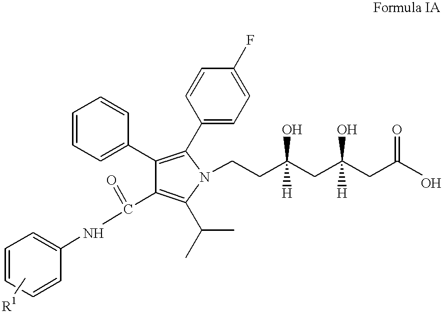
wherein R¹ is hydrogen or hydroxy and a pharmaceutically acceptable carrier, vehicle or diluent, said first pharmaceutical composition comprising of [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and a pharmaceutically acceptable carrier, vehicle or diluent.

20. A first pharmaceutical composition for use with a second pharmaceutical composition for achieving a therapeutic effect in a mammal, which effect is greater than the individual therapeutic effects achieved by admistering said first or second pharmaceutical compositions separately and which second pharmaceutical composition comprises an amount of [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester and a pharmaceutically acceptable carrier, vehicle or diluent, said first pharmaceutical composition comprising an amount of a compound having the Formula I 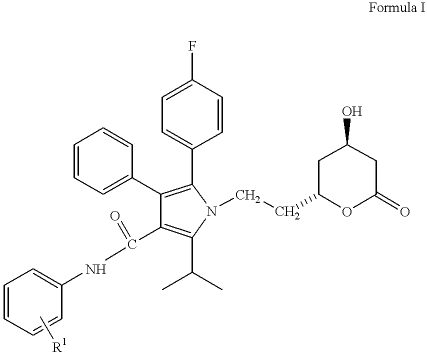
or, the open chain Formula IA 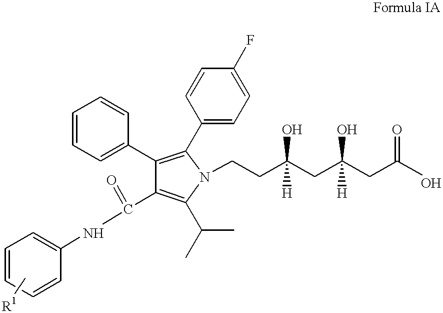
wherein R¹ is hydrogen or hydroxy or the pharmaceutically acceptable salts thereof and a pharmaceutically acceptable carrier, vehicle or diluent.
